SEQUENCE LISTING (26 sequences)

SEQ ID NO: 1 - protein, 463 aa, Clostridium difficile
  source 1..463: note = strain CCUG 20309
MKKFRKHKRISNCISILLILYLTLGGLLPNNIYAQDLQSYSEKVCNTTYKAPIERPEDFLKDKEKAKEWERKEAERIEQKLERSEKEALESYKKDSVEISKYSQTRNYFYDYQIEANSREKEYKELRNAISKNKIDKPMYVYYFESPEKFAFNKVIRTENQNEISLEKFNEFKETIQNKLFKQDGFKDISLYEPGKGDEKPTPLLMHLKLPRNTGMLPYTNTNNVSTLIEQGYSIKIDKIVRIVIDGKHYIKAEASVVSSLDFKDDVSKGDSWGKANYNDWSNKLTPNELADVNDYMRGGYTAINNYLISNGPVNNPNPELDSKITNIENALKREPIPTNLTVYRRSGPQEFGLTLTSPEYDFNKLENIDAFKSKWEGQALSYPNFISTSIGSVNMSAFAKRKIVLRITIPKGSPGAYLSAIPGYAGEYEVLLNHGSKFKINKIDSYKDGTITKLIVDATLIP

SEQ ID NO: 2 - protein, 463 aa, Clostridium difficile; identical to SEQ ID NO: 1
  source 1..463: note = strain CD196

SEQ ID NO: 3 - protein, 463 aa, Clostridium difficile
  source 1..463: note = strain AM478
MKKFRKYKSISNCISISLILYLTLGSLLPNNIYAQDLQSYSEKVYNTTYKAPIERPEDFLKDKEKAKEWERKEAERIEQKLERSEKEALESYKKDSVEINKYSQTRNYFYDYQIEANSREKEYRELRNAISKNKIDKPMYVYYFESPEKFAFNKVIRTENQNEISLEKFNEFKETIQDKLFKQDGFKDISLYEPGKGDEKPTPLLMHLKLPRNTGMLPYTNTNNISTLIEQGYSIKIDKIVRIVIDGKHYIKAEASVVNSLDFKDDVSKGDSWGKANYSDWSNKLTPNELADVNDYMRGGYTAINNYLISNGPVNNPNSELDSKITNIENALKREPIPTNLTVYRRSGPQEFGLTLTSPEYDFNKPENIDAFKSKWEGQTLSYPNFISTSIGSVNMSAFAKRKIVLRITIPKGSPGAYLSAIPGYAGEYEVLLNHGSKFKISKIDSYKDGAITKLIVDATLIP

SEQ ID NO: 4 - protein, 463 aa, Clostridium difficile
  source 1..463: note = strain CD98
MKKFRKHKSISNCISILLILYLTLGSLLPNNIYAQDLQSYSEKVCNTTYKAPIERPEDFLKDKERAKEWERKEAERIEQKLERSEKEALESYKKDSVEINKYSQTRNYFYDYQIEANSREKEYRELRNAISKNKIDKPMYVYYFESPEKFAFNKVIRTENQNEISLEKFNEFKETIQNKLFKQDGFKEISLYEPGKGDEEPTPLLMHLKLPRNTGMLPYTNTNNVSTLIEQGYSIKIDKIVRIVIDGKHYIKAEASVVSSLDFKDDVSKGDSWGKANYNDWSNKLTPNELADVNDYMRGGYTAINNYLISNGPVNNPNPELDSKITNIENALKREPIPTNLTVYRRSGPQEFGLTLTSPEYDFNKPENIDAFKSKWEGQTLSYPNFISTSIGSVNMSAFAKRKIVLRITIPKGSPGAYLSAIPGYAGEYEVLLNHGSKFKISKIDSYKDGAITKLIVDATLIP

SEQ ID NO: 5 - protein, 463 aa, Clostridium difficile
  source 1..463: note = strain AE16
MKKFRKHKRISNCISILLILYLTLGGLLPNNIYAQDLQSYSEKVCNTTYKAPIERPEDFLKDKEKAKEWERKEAERIEQKLERSEKEALESYKKDSVEISKYSQTRNYFYDYQIEANSREKEYKELRNAISKNKIDKPMYVYYFESPEKFAFNKVIRTENQNEISLEKFNEFKETIQNKLFKQDGFKDISLYEPGKGDEKPTPLLMHLKLPRNTGMLPYTNTNNVSTLIEQGYSIKIDKIVRIVIDGKHYIKAEASVVSSLDFKDDVSKGDSWGKANYNDWSNKLTPNELADVNDYMRGGYTAINNYLISNGPVVNPNPELDSKITNIENALKREPIPTNLTVYRRSGPQEFGLTLTSPEYDFNKLENIDAFKSKWEGQALSYPNFISTSIGSVNMSAFAKRKIVLRITIPKGSPGAYLSAIPGYAGEYEVLLNHGSKFKINKIDSYKDGTITKLIVDATLIP

SEQ ID NO: 6 - protein, 463 aa, Clostridium difficile
  source 1..463: note = strain CD196
MKKFRKHKRISNCISILLILYLTLGGLLPNNIYAQDLQSYSEKVCNTTYKAPIERPEDFLKDKEKAKEWERKEAERIEQKLERSEKEALESYKKDSVEISKYSQTRNYFYDYQIEANSREKEYKELRNAISKNKIDKPMYVYYFESPEKFAFNKVIRTENQNEISLEKFNEFKETIQNKLFKQDGFKDISLYEPGKGDEKPTPLLMHLKLPRNTGMLPYTNTNNVSTLIEQGYSIKIDKIVRIVIDGKHYIKAEASVVNSLDFKDDVSKGDSWGKANYNDWSNKLTPNELADVNDYMRGGYTAINNYLISNGPVNNPNPELDSKITNIENALKREPIPTNLTVYRRSGPQEFGLTLTSPEYDFNKLENIDAFKSKWEGQALSYPNFISTSIGSVNMSAFAKRKIVLRITIPKGSPGAYLSAIPGYAGEYEVLLNHGSKFKINKIDSYKDGTITKLIVDATLIP

SEQ ID NO: 7 - protein, 463 aa, Clostridium difficile
  source 1..463: note = strain A765
MKKFRKHKRISNCIFILLILYLTLGGLLPNNIYAQDLQSYSEKVCNTTYKAPIERPEDFLKDKEKAKEWERKEAERIEQKLERSEKEALESYKKDSVEISKYSQTRNYFYDYQIEANSREKEYKELRNAISKNKIDKPMYVYYFESPEKFAFNKVIRTENQNEISLEKFNEFKETIQNKLFKQDGFKDISLYEPGKGDEKPTPLLMHLKLPRNTGMLPYTNTNNVSTLIEQGYSIKIDKIVRIVIDGKHYIKAEASVVSSLDFKDDVSKGDSWGKANYNDWSNKLTPNELADVNDYMRGGYTAINNYLISNGPVNNPNPELDSKITNIENALKREPIPTNLTVYRRSGPQEFGLTLTSPEYDFNKLENIDAFKSKWEGQALSYPNFISTSIGSVNMSAFAKRKIVLRITIPKGSPGAYLSAIPGYAGEYEVLLNHGSKFKINKIDSYKDGTITKLIVDATLIP

SEQ ID NO: 8 - protein, 463 aa, Clostridium difficile; identical to SEQ ID NO: 1
  source 1..463: note = strain CCUG 20309

SEQ ID NO: 9 - protein, 463 aa, synthetic construct
MKKFRKXKXISNCIXIXLILYLTLGXLLPNNIYAQDLQSYSEKVXNTTYKAPIERPEDFLKDKEXAKEWERKEAERIEQKLERSEKEALESYKKDSVEIXKYSQTRNYFYDYQIEANSREKEYXELRNAISKNKIDKPMYVYYFESPEKFAFNKVIRTENQNEISLEKFNEFKETIQXKLFKQDGFKXISLYEPGKGDEXPTPLLMHLKLPRNTGMLPYTNTNNXSTLIEQGYSIKIDKIVRIVIDGKHYIKAEASVVXSLDFKDDVSKGDSWGKANYXDWSNKLTPNELADVNDYMRGGYTAINNYLISNGPVXNPNXELDSKITNIENALKREPIPTNLTVYRRSGPQEFGLTLTSPEYDFNKXENIDAFKSKWEGQXLSYPNFISTSIGSVNMSAFAKRKIVLRITIPKGSPGAYLSAIPGYAGEYEVLLNHGSKFKIXKIDSYKDGXITKLIVDATLIP

SEQ ID NO: 10 - protein, 876 aa, Clostridium difficile
  source 1..876: note = strain CCUG 20309
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSNIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 11 - protein, 876 aa, Clostridium difficile
  source 1..876: note = strain CD196
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSNIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNQDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIMDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 12 - protein, 876 aa, Clostridium difficile
  source 1..876: note = strain CD98
MKIQMRNKKVLSFLTLTAIVSQALAYPVYAQTSTSSHSDNKKEIINEDILTNNGLMGYYFTDEHFKDLKLMAPIKDGNLKFEEKKVDKLLNKDKSNVKSIRWTGRIIPSKDGEYTLSTDRDDILMQVNNESTISNTLKVNMKKGKEYKFRIELQDKNLGSIDNLSSPNLYWELDGIKKIIPAENLFLRDYSNIEKNDPFIPNNNFFDPRLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQNPEDKTPELTIGEAIEKAFGATKKDGLLYFNDIPIDESCVELIFDDNIGNKIKDSLKTLSDKQICNVKLERGMNILIKTPTYFTNFDDYNNYPSTWSNVNTTNQDGLQGSANKLNGETKIKIPMSKLKPYKRYVFSGYSKDPLTSNSIIVKIKAKEEKTDYLVPEQGYTKFSYEFETTEKDSSNIEITLIGSGTTYLDNLSITELNSTPEILNEPEVKIPTDQEIIDAHKIYSADLNFNPSTGNAYINGMYFTPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 13 - protein, 876 aa, Clostridium difficile
  source 1..876: note = strain K744
MKVQMRNKKVLSFLTLTAIVSQALAYPVYAQTSTSSHSDNKKEIINEDILTNNGLMGYYFTDEHFKDLKLMAPIKDGNLKFEEKKVDKLLNKDKSNVKSIRWTGRIIPSKDGEYTLSTDRDDILMQVNNESTISNTLKVNMKKGKEYKFRIELQDKNLGSIDNLSSPNLYWELDGIKKIIPAENLFLRDYSNIEKNDPFIPNNNFFDPRLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQNPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNQDGLQGSANKLNGETKIKIPMSKLKPYKRYVFSGYSKDPLTSNSIIVKIKAKEEKTDYLVPEQGYTKFSYEFETTEKDSSNIEITLIGSGTTYLDNLSITELNSTPEILNEPEVKIPTDQEIIDAHKIYSADLNFNPSTGNAYINGMYFTPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 14 - protein, 876 aa, Clostridium difficile
  source 1..876: note = strain AM478
MKVQMRNKKVLSFLTFTAIVSQALVYPVYAQTSTSNHSNKKKEIINEDILPNNGLMGYYFTDEHFKDLKLMAPIKDGNLKFEEKKVDKLLDKDKSDVKSIRWTGRIIPSKDGEYTLSTDRDDVLMQVNTESTISNKLKVNMKKGKEYKVRIELQDKNLGSIDNLSSPNLYWELDGIKKIIPAENLFLRDYSNIEKNDPFIPNNNFFDPRLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNFQDPEDKTPELTIGEAIEKAFGATKKDGLLYFNDIPIDESCVELIFDDNTANKIKDSLKTLSDKQIYNVKLERGMNILIKTPTYFINFDDYNNYPSIWSNVNTTNQDGLQGSANKLNGETKIKIPMSELKPYKRYVFSGYSKDPLTSNSIIVKIKAKEEKTDFLVPEQEYTKFSYEFETTEKDSSNIEITLIGSGTTYLDNLSITELNSTPEILNEPEVKIPTDQEIIDAHKIYSADLNFNPSTGNAYINGMYFTPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 15 - protein, 876 aa, Clostridium difficile
  source 1..876: note = strain AD667
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSNIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPYDRELLVLSVD

SEQ ID NO: 16 - protein, 876 aa, Clostridium difficile
  source 1..876: note = strain C421
MKIQMRNKKVLSFLTLTAIVSQALVYPVYAQTSTSNHSNKKKEIVNEDILPNNGLMGYYFTDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSTIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNKDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIIDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 17 - protein, 876 aa, Clostridium difficile; identical to SEQ ID NO: 10
  source 1..876: note = strain A765

SEQ ID NO: 18 - protein, 876 aa, Clostridium difficile
  source 1..876: note = strain CD196
MKIQMRNKKVLSFLTLTAIVSQALVYPVYAQTSTSNHSNKKKEIVNEDILPNNGLMGYYFSDEHFKDLKLMAPIKDGNLKFEEKKVDKLLDKDKSDVKSIRWTGRIIPSKDGEYTLSTDRDDVLMQVNTESTISNTLKVNMKKGKEYKVRIELQDKNLGSIDNLSSPNLYWELDGMKKIIPEENLFLRDYSNIEKDDPFIPNNNFFDPKLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNLQDPEDKTPELTIGEAIEKAFGATKKDGLLYFNDIPIDESCVELIFDDNTANKIKDSLKTLSDKKIYNVKLERGMNILIKTPTYFTNFDDYNNYPSTWSNVNTTNQDGLQGSANKLNGETKIKIPMSELKPYKRYVFSGYSKDPLTSNSIIVKIKAKEEKTDYLVPEQGYTKFSYEFETTEKDSSNIEITLIGSGTTYLDNLSITELNSTPEILDEPEVKIPTDQEIMDAHKIYFADLNFNPSTGNTYINGMYFAPTQTNKEALDYIQKYRVEATLQYSGFKDIGTKDKEMRNYLGDPNQPKTNYVNLRSYFTGGENIMTYKKLRIYAITPDDRELLVLSVD

SEQ ID NO: 19 - protein, 876 aa, synthetic construct
MKXQMRNKKVLSFLTXTAIVSQALXYPVYAQTSTSXHSXXKKEIXNEDILXNNGLMGYYFXDEHFKDLKLMAPIKDGNLKFEEKKVDKLLXKDKSXVKSIRWTGRIIPSKDGEYTLSTDRDDXLMQVNXESTISNXLKVNMKKGKEYKXRIELQDKNLGSIDNLSSPNLYWELDGXKKIIPXENLFLRDYSXIEKXDPFIPNNNFFDPXLMSDWEDEDLDTDNDNIPDSYERNGYTIKDLIAVKWEDSFAEQGYKKYVSNYLESNTAGDPYTDYEKASGSFDKAIKTEARDPLVAAYPIVGVGMEKLIISTNEHASTDQGKTVSRATTNSKTESNTAGVSVNVGYQNGFTANVTTNYSHTTDNSTAVQDSNGESWNTGLSINKGESAYINANVRYYNTGTAPMYKVTPTTNLVLDGDTLSTIKAQENQIGNNLSPGDTYPKKGLSPLALNTMDQFSSRLIPINYDQLKKLDAGKQIKLETTQVSGNFGTKNSSGQIVTEGNSWSDYISQIDSISASIILDTENESYERRVTAKNXQXPEDKTPELTIGEAIEKAFGATKKDGLLYFNDIPIDESCVELIFDDNXXNKIKDSLKTLSDKXIXNVKLERGMNILIKTPTYFXNFDDYNNYPSXWSNVNTTNXDGLQGSANKLNGETKIKIPMSXLKPYKRYVFSGYSKDPLTSNSIIVKIKAKEEKTDXLVPEQXYTKFSYEFETTEKDSSNIEITLIGSGTTYLDNLSITELNSTPEILXEPEVKIPTDQEIXDAHKIYXADLNFNPSTGNXYINGMYFXPTQTNKEALDYIQKYRVEATLQYSGFKDIGTKDKEMRNYLGDPNQPKTNYVNLRSYFTGGENIMTYKKLRIYAITPXDRELLVLSVD

SEQ ID NO: 20 - protein, 719 aa, Clostridium difficile
  source 1..719: note = strain 630
MNKKNIAIAMSGLTVLASAAPVFAATTGTQGYTVVKNDWKKAVKQLQDGLKDNSIGKITVSFNDGVVGEVAPKSANKKADRDAAAEKLYNLVNTQLDKLGDGDYVDFSVDYNLENKIITNQADAEAIVTKLNSLNEKTLIDIATKDTFGMVSKTQDSEGKNVAATKALKVKDVATFGLKSGGSEDTGYVVEMKAGAVEDKYGKVGDSTAGIAINLPSTGLEYAGKGTTIDFNKTLKVDVTGGSTPSAVAVSGFVTKDDTDLAKSGTINVRVINAKEESIDIDASSYTSAENLAKRYVFDPDEISEAYKAIVALQNDGIESNLVQLVNGKYQVIFYPEGKRLETKSANDTIASQDTPAKVVIKANKLKDLKDYVDDLKTYNNTYSNVVTVAGEDRIETAIELSSKYYNSDDKNAITDKAVNDIVLVGSTSIVDGLVASPLASEKTAPLLLTSKDKLDSSVKSEIKRVMNLKSDTGINTSKKVYLAGGVNSISKDVENELKNMGLKVTRLSGEDRYETSLAIADEIGLDNDKAFVVGGTGLADAMSIAPVASQLKDGDATPIVVVDGKAKEISDDAKSFLGTSDVDIIGGKNSVSKEIEESIDSATGKTPDRISGDDRQATNAEVLKEDDYFTDGEVVNYFVAKDGSTKEDQLVDALAAAPIAGRFKESPAPIILATDTLSSDQNVAVSKAVPKDGGTNLVQVGKGIASSVINKMKDLLDM

SEQ ID NO: 21 - protein, 756 aa, Clostridium difficile
  source 1..756: note = strain 1 (R8366)
MNKKNIAIAMSGLTVLASAAPVFADDTKVETGDQGYTVVQSKYKKAVEQLQKGILDGSITEIKVFFEGTLASTIKVGSELNAADASKLLFTQVDNKLDNLGDGDYVDFLITSPGQGDKITTSKLVALKDLTGASADAIIAGTSSADGVVTNTGAASGSTETNSAGTKLAMSAIFDTAYTDSSETAVKITIKADMNDTKFGKAGETTYSTGLTFEDGSTEKIVKLGDSDIIDITKALKLTVVPGSKATVKFAEKTPSASVQPVITKLRIINAKEETIDIDASSSKTAQDLAKKYVFNKTDLNTLYKVLNGDEADTNGLIEEVSGKYQVVLYPEGKRVTTKSAAKASIADENSPVKLTLKSDKKKDLKDYVDDLRTYNNGYSNAIEVAGEDRIETAIALSQKYYNSDDENAIFRDSVDNVVLVGGNAIVDGLVASPLASEKKAPLLLTSKDKLDSSVKAEIKRVMNIKSTTGINTSKKVYLAGGVNSISKEVENELKDMGLKVTRLAGDDRYETSLKIADEVGLDNDKAFVVGGTGLADAMSIAPVASQLRNANGKMDLADGDATPIVVVDGKAKTINDDVKDFLDDSQVDIIGGENSVSKDVENAIDDATGKSPDRYSGDDRQATNAKVIKESSYYQDNLNNDKKVVNFFVAKDGSTKEDQLVDALAAAPVAANFGVTLNSDGKPVDKDGKVLTGSDNDKNKLVSPAPIVLATDSLSSDQSVSISKVLDKDNGENLVQVGKGIATSVINKLKDLLSM

SEQ ID NO: 22 - protein, 714 aa, Clostridium difficile
  source 1..714: note = strain 17 (R7404)
MNKKNLAMAMAAVTVVGSAAPIFADSTTPGYTVVKNDWKKAVKQLQDGLKNKTISTIKVSFNGNSVGEVTPASSGAKKADRDAAAEKLYNLVNTQLDKLGDGDYVDFEVTYNLATQIITKAEAEAVLTKLQQYNDKVLINSATDTVKGMVSDTQVDSKNVAANPLKVSDMYTIPSAITGSDDSGYSIAKPTEKTTSLLYGTVGDATAGKAITVDTASNEAFAGNGKVIDYNKSFKATVQGDGTVKTSGVVLKDASDMAATGTIKVRVTSAKEESIDVDSSSYISAENLAKKYVFNPKEVSEAYNAIVALQNDGIESDLVQLVNGKYQVIFYPEGKRLETKSADIIADADSPAKITIKANKLKDLKDYVDDLKTYNNTYSNVVTVAGEDRIETAIELSSKYYNSDDKNAITDDAVNNIVLVGSTSIVDGLVASPLASEKTAPLLLTSKDKLDSSVKSEIKRVMNLKSDTGINTSKKVYLAGGVNSISKDVENELKNMGLKVTRLSGEDRYETSLAIADEIGLDNDKAFVVGGTGLADAMSIAPVASQLKDGDATPIVVVDGKAKEISDDAKSFLGTSDVDIIGGKNSVSKEIEESIDSATGKTPDRISGDDRQATNAEVLKEDDYFKDGEVVNYFVAKDGSTKEDQLVDALAAAPIAGRFKESPAPIILATDTLSSDQNVAVSKAVPKDGGTNLVQVGKGIASSVINKMKDLLDM

SEQ ID NO: 23 - protein, 361 aa, synthetic construct
MNKKNXAXAMXXXTVXXSAAPXFAXXXXXXTXXXGYTVVXXXXKKAVXQLQXGXXXXXIXXIXVXFXXXXXXXXXXXSXXXXXADXXXAXXXLXXXVXXXLDXLGDGDYVDFXXXXXXXXXXITXXXXXXXXXTXKLXXXXXXXXXXXXXIXXXTXXXXGXVXXTXXXXXXXXXXXXXXKXXXXXXXXXXXXXSXXXXXXXXXXXXXXXXXXXGXXGXXTXXXXXXXXXXXXEXXXXXGXXXXIDXXKXXKXXVXXXXXXXXXXXXXXXXKXXXXXXXXXXXXXRXXXAKEEXIDXDXSSXXXAXXLAKXYVFXXXXXXXXYXXXXXLXXDXXXXXXLXXXVXGKYQVXXYPEGKRXXTKS

SEQ ID NO: 24 - protein, 298 aa, synthetic construct
MNKKNXAXAMXXXTVXXSAAPXFAXXXXXXTXXXGYTVVXXXXKKAVXQLQXGXXXXXIXXIXVXFXXXXXXXXXXXSXXXXXADXXXAXXXLXXXVXXXLDXLGDGDYVDFXXXXXXXXXXITXXXXXXXXXTXKLXXXXXXXXXXXXXIXXXTXXXXGXVXXTXXXXXXXXXXXXXXKXXXXXXXXXXXXXSXXXXXXXXXXXXXXXXXXXGXXGXXTXXXXXXXXXXXXEXXXXXGXXXXIDXXKXXKXXVXXXXXXXXXXXXXXXXKXXXXXXXXXXXXXRXXXAKEEXIDXDX

SEQ ID NO: 25 - protein, 63 aa, synthetic construct
SSXXXAXXLAKXYVFXXXXXXXXYXXXXXLXXDXXXXXXLXXXVXGKYQVXXYPEGKRXXTKS

SEQ ID NO: 26 - protein, 416 aa, synthetic construct
AXXXXIAXXXXPXKXXXKXXKXKDLKDYVDDLXTYNNXYSNXXXVAGEDRIETAIXLSXKYYNSDDXNAIXXXXVXXXVLVGXXXIVDGLVASPLASEKXAPLLLTSKDKLDSSVKXEIKRVMNXKSXTGINTSKKVYLAGGVNSISKXVENELKXMGLKVTRLXGXDRYETSLXIADEXGLDNDKAFVVGGTGLADAMSIAPVASQLXXXXXXXXXXDGDATPIVVVDGKAKXIXDDXKXFLXXSXVDIIGGXNSVSKXIEXXIDXATGKXPDRXSGDDRQATNAXVXKEXXYXXDXXXXXXXVVNXFVAKDGSTKEDQLVDALAAAPXAXXFXXXXXXXXXXXXXXXXXXXXXXXXXXXXXSPAPIXLATDXLSSDQXVXXSKXXXKDXGXNLVQVGKGIAXSVINKXKDLLXM

(19) United States
(12) Patent Application Publication
(10) Pub. No.: US 2026/0232785 A1
(43) Pub. Date: Aug. 13, 2026

(54) VACCINES AGAINST CLOSTRIDIOIDES DIFFICILE AND METHODS OF USING THE SAME

(71) Applicant: CLOSTRIDIAL DISEASE PREVENTION INC., Greenville, DE (US)

(72) Inventor: Joseph M. Sasser, Cutler Bay, FL (US)

(21) Appl. No.: 19/330,601

(22) Filed: Sep. 16, 2025

Related U.S. Application Data
(60) Provisional application No. 63/739,541, filed on Dec. 28, 2024.
(60) Provisional application No. 63/744,813, filed on Jan. 13, 2025.
(60) Provisional application No. 63/800,824, filed on May 6, 2025.
(60) Provisional application No. 63/800,748, filed on May 6, 2025.
(60) Provisional application No. 63/854,874, filed on Jul. 31, 2025.
(60) Provisional application No. 63/859,298, filed on Aug. 7, 2025.
(60) Provisional application No. 63/860,823, filed on Aug. 9, 2025.

(51) Int. Cl. A61K 39/08; A61K 39/00; A61P 1/16; A61P 35/00; A61P 37/04

(52) U.S. Cl. CPC A61K 39/08; A61P 1/16; A61P 35/00; A61P 37/04; A61K 2039/70; A61K 2039/82

(57) ABSTRACT

Disclosed are vaccine compositions comprising polypeptides selected from Clostridioides difficile binary toxin B, Clostridioides difficile binary toxin A, a High Molecular Weight portion of Clostridioides difficile Surface Layer Protein A, and a Low Molecular Weight portion of Clostridioides difficile Surface Layer Protein A. Methods of preparing the vaccine composition, methods of preventing or reducing attachment of C. difficile to an epithelial cell, and methods of preventing or treating a condition caused by infection of Clostridioides difficile are also provided.

### CROSS-REFERENCE TO RELATED APPLICATIONS

This application claims the benefit of U.S. Provisional Application Ser. No. 63/860,823, filed Aug. 9, 2025, U.S. Provisional Application Ser. No. 63/854,874, filed Jul. 31, 2025, U.S. Provisional Application Ser. No. 63/800,824, filed May 6, 2025, U.S. Provisional Application Ser. No. 63/800,748, filed May 6, 2025, U.S. Provisional Application Ser. No. 63/744,813, filed Jan. 13, 2025, U.S. Provisional Application Ser. No. 63/859,298, filed Aug. 7, 2025, and U.S. Provisional Application Ser. No. 63/739,541, filed Dec. 28, 2024, the entire contents of each of which are incorporated by reference in their entirety.

### SEQUENCE LISTING

The contents of the electronic sequence listing (18648300002.xml; Size: 43,028 bytes; and Date of Creation: Sep. 16, 2025) is herein incorporated by reference in its entirety.

### BACKGROUND

Clostridioides difficile (C. difficile) infection (CDI) is a serious bacterial infection which causes inflammation of the colon, leading to symptoms like diarrhea, fever, abdominal pain, and in severe cases, life-threatening complications. The CDC estimates that 30,000 deaths are directly attributable to the disease and annual costs exceed $5 billion, including expenses related to hospitalization, treatment, and lost productivity. Additionally, C. difficile can cause or lead to the development of numerous other diseases, disorders, and conditions, such as high cholesterol, heart attack, stroke, irritable bowel syndrome, metabolic dysfunction-associated steatoic liver disease, metabolic dysfunction-associated steatohepatitis, and colorectal cancer.

Antibiotic use is the common treatment for C. difficile, but paradoxically can lead to an increase in CDI, by reducing overall gut microflora. This dysbiosis can lead dormant C. difficile populations that have colonized the colon (i.e. the spore form) to shift to vegetative and germinating C. diff cells, which take advantage of the reduced competition for nutrients. Another approach being tested is a vaccine candidate against C. difficile Toxin A and Toxin B, the primary causes of CDI chronic diarrhea. However, these vaccine candidates did not meet their clinical endpoints, and both were abandoned. Accordingly, there is a need for vaccines to prevent CDI and rCDI.

### SUMMARY

In a first aspect, disclosed herein are vaccine compositions comprising at least two polypeptides selected from the group consisting of a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP). In embodiments, the at least two polypeptides are CDTb and HMW SLP.

In a second aspect, disclosed herein are methods of preparing vaccine compositions comprising at least two polypeptides selected from the group consisting of a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP). The method comprises expressing a polynucleotide encoding the at least two polypeptides.

In a third aspect, disclosed herein are methods of preventing or reducing attachment of Clostridioides difficile (C. difficile) to an epithelial cell in a subject in need thereof comprising administering a vaccine composition comprising at least two polypeptides selected from the group consisting of a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP) to the subject.

In a fourth aspect, disclosed herein are methods of preventing or treating a disease, disorder, or condition caused by infection of Clostridioides difficile comprising administering a vaccine composition comprising at least two polypeptides selected from the group consisting of a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP). In embodiments, the disease, disorder, or condition is selected from the group consisting of chronic diarrhea, high cholesterol, heart attack, stroke, irritable bowel syndrome, metabolic dysfunction-associated steatoic liver disease, metabolic dysfunction-associated steatohepatitis, and colorectal cancer.

### BRIEF DESCRIPTION OF THE DRAWINGS

FIG. 1. depicts the cholesterol synthesis pathway.

### DETAILED DESCRIPTION

Disclosed are novel vaccine compositions comprising polypeptides of Clostridioides difficile (C. difficile) binary toxin and Surface Layer Protein A (SLP). The C. difficile binary toxin includes a C. difficile binary toxin B (CDTb) and a C. difficile binary toxin A (CDTa). The C. difficile SLP includes a High Molecular Weight portion (HMW SLP), and a Low Molecular Weight portion (LMW SLP). Methods of preparing the vaccine composition, methods of preventing or reducing attachment of C. difficile to an epithelial cell, and methods of preventing or treating a condition caused by infection of C. difficile are also provided.

The inventor has identified four polypeptides, CDTa, CDTb, HMW SLP, and LMW SLP, that in novel combinations can provide a vaccine composition, and the vaccine composition can be administered to a subject to prevent conditions associated with C. difficile infection (CDI). The vaccine composition can greatly reduce the bacteria's attachment to and in a cell, which can also minimize the release of C. difficile's pathogenic Toxins A and B into host cells. These effects of the vaccine composition can lead to prevention and/or reduction in severity of C. difficile infection and/or recurrent infection (rCDI). CDI and rCDI are primary causes of chronic diarrhea. Previous “unsuccessful” vaccines were targeted at reducing Toxin A and Toxin B only. In those studies, when the population of C. difficile spores was large, spores remained after treatment. As a result, C. difficile continued to germinate and continued to produce Toxin A and Toxin B, which led to recurrent infection. The novel vaccine compositions provided in this disclosure can reduce the population of C. difficile and C. difficile spores in the colon by targeting the CDT and SLP proteins. As C. difficile uses CDT and SLP proteins to obtain nutrients from the colon cells, these proteins are a more effective vaccine target than vaccines that target Toxin A and Toxin B.

### Vaccine Composition

In a first aspect, this disclosure provides vaccine compositions comprising at least two polypeptides selected from a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP). As used herein, the term “vaccine composition” refers to a preparation including an immunogen (e.g. the polypeptide described herein) derived from a pathogen, which is used to induce an immune response against the pathogen that provides protective immunity (e.g., immunity that protects a subject against infection with the pathogen and/or reduces the severity of the disease, such aschronic diarrhea, caused by infection with the pathogen). The protective immune response may include formation of antibodies and/or a cell-mediated response.

C. difficile produces three toxins: Toxin A, Toxin B, and binary toxin C. difficile transferase (CDT), which is composed of CDTa and CDTb. Toxin A and Toxin B are the two main pathogenic toxins of C. difficile infection. A third toxin, Binary Toxin, also known as CDT, is also produced by the bacteria. CDT is a binary toxin comprising two components. One component, CDTa, is the biologically active component and possesses ADP-ribosyltransferase activity to modify actin. The second component, CDTb, is involved in binding of the toxin to host cells and is responsible for the translocation of the enzyme component into the cytosol (Gerding et al., Clostridium difficile binary toxin CDT: mechanism, epidemiology, and potential clinical importance. Gut Microbes. 2014 January-February; 5(1):15-27. doi: 10.4161/gmic.26854. Epub 2013 October 31. PMID: 24253566; PMCID: PMC4049931). CDTa is the enzymatic ADP-ribosyltransferase that modifies actin, and CDTb binds to host cells and translocates CDTa into the cytosol. CDTb is activated by serine proteases and binds to lipolysis stimulated lipoprotein receptor. ADP-ribosylation induces depolymerization of the actin cytoskeleton. Toxin-induced actin depolymerization also produces microtubule-based membrane protrusions, which form a network on epithelial cells and increase bacterial adherence. CDT is frequently observed in C. difficile strains associated with increased severity of CDI. Sequences encoding CDTa and CDTb are known in the art. Exemplary sequences for CDTa and CDTb are disclosed herein, including SEQ ID NOs: 1-8 and 10-18. There are eight exemplary sequences for CDTa disclosed herein that come from various C. difficile strains, including: CCUG 20309, CD196, AM478m, CD98, A765, AE16. The eight exemplary sequences for CDTa have about 95% sequence identity amongst each other. There are nine exemplary sequences for CDTb disclosed herein that come from various C. difficile strains, including: CCUG 20309, CD196, AM478, CD98, A765, K744, AD667, and C421. The nine exemplary sequences for CDTb also have about 95% sequence identity amongst each other. The exemplary sequences for CDTa and CDTb were aligned to create a consensus sequence for each protein, SEQ ID NOs: 9 and 19, respectively. Amino acid residues indicated by an “X” may be any naturally occurring or non-naturally occurring amino acid.

In addition to the three toxins described above, C. difficile has a precursor surface layer protein (SLP) that, when cleaved, forms two mature proteins that facilitate adherence to host cells: a conserved high molecular weight SLP and a low molecular weight SLP. A single gene, slpA, that has a conserved genomic location among all C. difficile strains, encodes the precursor protein, termed SlpA. SlpA comprises four distinct segments: A signaling peptide segment, an LMW SLP segment, an HMW SLP segment, and a linker between the LMW SLP and the HMW SLP. (Mori, N and Takahashi, T. Characteristics and Immunological Roles of Surface Layer Proteins in Clostridium difficile. Ann Lab Med., 2018 May; 38(3):189-195. doi: 10.3343/alm.2018.38.3.189. PMID: 29401552; PMCID: PMC5820062). SlpA as cleaved generates the two mature proteins, HMW SLP and LMW SLP. Sequences encoding the SlpA precursor protein, the HMW SLP, and the LMW SLP are known in art. An exemplary sequence of the SlpA precursor protein is disclosed herein. The exemplary sequence of the SlpA precursor protein is from C. difficile strain 630 (SEQ ID NO: 20), which is a reference strain of C. difficile. Consensus sequences for the LMW SLP (with and without the linker at SEQ ID NOs: 23 and 24, respectively), the linker (SEQ ID NO: 25), and the HMW SLP (SEQ ID NO: 26) are disclosed herein. The consensus sequences were generated by aligning the relevant portions of the exemplary sequence for the SlpA precursor protein from C. difficile strain 630 with two other strains, R8366 (accession no. CAC35721.1) (SEQ ID NO: 21) and R7404 (accession no. CAC35720.1) (SEQ ID NO: 22). (Calabi et al., Molecular characterization of the surface layer proteins from Clostridium difficile. Molecular Microbiology (2001) 40(5), 1187-1199). Amino acid residues indicated by an “X” may be any naturally occurring or non-naturally occurring amino acid.

In some aspects, the vaccine composition has two polypeptides, CDTb and HMW SLP. In embodiments, the amino acid sequence of CDTb may be selected from the group consisting of SEQ ID NOs: 10-19. In embodiments, the amino acid sequence of HMW SLP may comprise SEQ ID NO: 26. In some embodiments, the vaccine composition may further comprise CDTa and/or LMW SLP. In embodiments, the amino acid sequence of CDTa may be selected from the group consisting of SEQ ID NOs: 1-9. In embodiments, the amino acid sequence of the LMW SLP may comprise SEQ ID NO: 23 or 24.

The polypeptides of the vaccine composition can be combined to create a fusion protein. As used herein, the term “fusion protein” means a protein comprised of two or more polypeptides that are joined or fused, directly or indirectly via a linker. The linker is a peptide that joins the polypeptides at the amino terminus of one protein and the carboxy terminus of another protein, to form a single continuous polypeptide. The linker may be cleavable by a protease.

In some embodiments, the vaccine composition may further comprise an adjuvant. As used herein, the term “adjuvant” refers to a compound that, when used in combination with an immunogen, augments or otherwise alters or modifies the immune response induced against the immunogen. Modification of the immune response may include intensification or broadening the specificity of either or both antibody and cellular immune responses. An adjuvant can include one or more of a peptide or modified peptide, protein, carbohydrate, or small molecule. The disclosed vaccine compositions may include an adjuvant, in various aspects. In some embodiments, the adjuvant can include one or more peptide or modified peptide, protein, carbohydrate, or small molecule.

In some embodiments, disclosed herein are pharmaceutical compositions comprising a vaccine composition and a pharmaceutically acceptable carrier or excipient. Any pharmaceutically acceptable or suitable carrier or excipient may be used in the disclosed compositions. The term “carrier” refers to a diluent, excipient, or vehicle with which the pharmaceutical composition is administered. Pharmaceutically acceptable carriers are known in the art and include, but are not limited to, diluents, preservatives, solubilizers, emulsifiers, liposomes, and nanoparticles. Pharmaceutically acceptable carriers or excipients may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, and suspensions, including saline and buffered media. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should be selected according to the mode of administration. The compositions may include a pharmaceutical carrier, excipient, or diluent, which are nontoxic to the cell or subject being exposed thereto at the dosages and concentrations employed.

In some aspects, a polynucleotide encodes the vaccine composition described herein. The term “polynucleotide” refers to a polydeoxyribonucleotide (containing 2-deoxy-D-ribose), a polyribonucleotide (containing D-ribose), and to any other type of polynucleotide that is an N glycoside of a purine or pyrimidine base. Polynucleotide includes double- and single-stranded DNA, as well as double- and single-stranded RNA. Polynucleotide also refers to DNA or RNA of genomic, natural, or synthetic origin (which may be single-stranded or double-stranded and may represent the sense or the antisense strand). In some embodiments, the polynucleotide may be the native polynucleotide sequence. In embodiments, the polynucleotide may be codon-optimized, which provides expression of the vaccine composition. For example, the disclosed polynucleotide encodes CDTb, CDTa, HMW SLP, or LMW SLP, or combinations thereof, where the polynucleotide contains codons that are optimized for expression in a particular host.

In some embodiments, each polypeptide of the vaccine composition can be encoded by a separate polynucleotide. In embodiments, a single polynucleotide may encode the vaccine composition, thus each polypeptide in the vaccine composition. The polynucleotide that encodes the vaccine composition may also encode a linker. In embodiments the linker may join the polypeptides in the vaccine composition.

A polynucleotide described herein can be operably linked to a promoter. The polynucleotide operably linked to a promoter may be in an expression vector. As used herein, the term “promoter” refers to a DNA sequence that regulates the transcription of a polynucleotide. Typically, a promoter is a regulatory region that is capable of binding RNA polymerase and initiating transcription of a downstream sequence. However, a promoter may be located at the 5′ or 3′ end, within a coding region, or within an intron of a gene that it regulates. Promoters may be derived in their entirety from a native gene, may be composed of elements derived from multiple regulatory sequences found in nature, or may comprise synthetic DNA segments. It is understood by those skilled in the art that different promoters may direct the expression of a gene in different tissues or cell types, at different stages of development, or in response to different environmental conditions. A promoter is “operably linked” to a polynucleotide if the promoter is connected to the polynucleotide such that it may affect transcription of the polynucleotide.

The term “vector” refers to some means by which DNA or RNA can be introduced into a host. There are various types of vectors including virus, plasmids, bacteriophages, and cosmids. In some embodiments, a virus comprises the polynucleotide. As used herein, a “viral vector” refers to recombinant viral nucleic acid that has been engineered to express a heterologous polypeptide. The recombinant viral nucleic acid typically includes cis-acting elements for expression of the heterologous polypeptide. The recombinant viral nucleic acid typically is capable of being packaged into a virus that can infect a host cell. For example, the recombinant viral nucleic acid may include cis-acting elements for packaging. Preferably, the viral vector is not replication competent, is attenuated, or at least does not cause disease. The viral vector may naturally be non-pathogenic to the host. Alternatively, the viral vector may be genetically altered by modern molecular biological methods (e.g., restriction endonuclease and ligase treatment, and rendered less virulent than wild type), typically by deletion of specific genes.

In some embodiments, a modified cell comprises a polynucleotide as described herein. An expression vector comprising a polynucleotide as described herein may be introduced to a cell to create the modified cell.

### Methods of Preparing a Vaccine

In an aspect, a vaccine composition is prepared comprising polypeptides as described herein which, upon introduction into a host, will confer immunity to that host, in the event the host is subsequently challenged by the same microorganism (e.g., C. difficile), which produced the protein(s). In preferred embodiments, the vaccine composition is a vaccine comprising polypeptides as described herein and further comprising an excipient and/or diluent appropriate where a composition is to be administered to a subject in need of vaccination against developing disease caused by C. difficile. In embodiments, the polypeptides in the vaccine composition comprise at least two polypeptides selected from C. difficile CDTb, CDTa, HMW SLP, and LMW SLP. In some embodiments, the at least two polypeptides are CDTb and HMW SLP. The amino acid sequence of CDTb may be selected from the group consisting of SEQ ID NOs: 10-19. The amino acid sequence of HMW SLP may comprise SEQ ID NO: 26. The vaccine composition may further comprise CDTa, wherein the amino acid sequence of CDTa is selected from the group consisting of SEQ ID NOs: 1-9. The vaccine composition may also further comprise LMW SLP, wherein the amino acid sequence of the LMW SLP may comprise SEQ ID NO: 23 or 24.

The term “vaccine,” as used herein, refers to a composition that includes an antigen. Vaccine may also include a biological preparation that improves immunity to a particular disease. A vaccine may typically contain an agent, referred to as an antigen, that resembles a disease-causing microorganism, and the agent may often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters. Similarly, the disclosed vaccine compositions may be combined with vaccines against other pathogens, which could “boost” the immune responses to the pathogen of interest, by acting themselves as vaccine adjuvants. Adjuvants can be classified according to their physiochemical properties or mechanisms of action. The two major classes of adjuvants include compounds that directly act on the immune system such as bacterial toxins that stimulate immune responses, and molecules able to facilitate the presentation of antigens in a controlled manner and behaving as a carrier.

In another aspect, disclosed are methods of preparing the vaccine composition described herein. The disclosed methods comprise expressing a polynucleotide encoding the vaccine composition, or encoding at least two polypeptides described herein. In aspects, the polypeptides are selected from C. difficile CDTb, CDTa, HMW SLP, and LMW SLP. In some embodiments, the at least two polypeptides are CDTb and HMW SLP. The amino acid sequence of CDTb may be selected from the group consisting of SEQ ID NOs: 10-19. The amino acid sequence of HMW SLP may comprise SEQ ID NO: 26. The vaccine composition may further comprise CDTa, wherein the amino acid sequence of CDTa is selected from the group consisting of SEQ ID NOs: 1-9. The vaccine composition may further comprise LMW SLP, wherein the amino acid sequence of the LMW SLP may comprise SEQ ID NO: 23 or 24.

A polynucleotide encoding the at least two polypeptides may be expressed in C. difficile or a modified cell. The polynucleotide can be in an expression vector, wherein the method further comprises introducing an expression vector comprising the polynucleotide into a cell. The polynucleotide then expresses the at least two polypeptides in the modified cell. The expression vector can be introduced according to methods well known in the art. For example, introducing nucleic acids can be achieved by calcium phosphate co-precipitation, electroporation, microinjection, lipofection, and transfection employing polyamine transfection reagents.

In some embodiments, the method further comprises purifying the polypeptides. The polypeptides may be purified from C. difficile or a modified cell transformed with an expression vector expressing the polynucleotide encoding the polypeptides. The polypeptides can be purified using industry-standard techniques that are common with similar vaccines and similar bacteria, such as Tetanus vaccine. Methods of purifying polypeptides to synthesize vaccines are known by those in the art. Methods of purifying polypeptides include, but are not limited to, centrifugation and chromatography, such as gel filtration chromatography, ion exchange chromatography, and affinity chromatography.

In some embodiments, the method further comprises inactivating the polypeptides. The polypeptides can be inactivated using industry-standard techniques that are in common with similar vaccines and similar bacteria, such as Tetanus vaccine. Methods of inactivating polypeptides to synthesize vaccines are known by those in the art. Methods of inactivating polypeptides include but are not limited to treatment with formaldehyde or solutions comprising formaldehyde, heat treatment, radiobiological techniques, genetic inactivation, or protein engineering. In some embodiments, the at least two polypeptides are inactivated by formaldehyde or a solution comprising formaldehyde, such as formalin.

Selection of appropriate vaccine components is within the routine capability of the skilled person. For example, the disclosed vaccine composition may conveniently be formulated using a pharmaceutically acceptable carrier, excipient or diluent, such as, for example, an aqueous solvent, non-aqueous solvent, non-toxic-excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the vaccine composition are adjusted according to routine skills.

### Methods of Using the Vaccine

In an aspect, methods of vaccinating a subject are disclosed. The methods can include administering to a subject a vaccine composition disclosed herein and/or one or more of the polynucleotides that encode the vaccine composition as disclosed herein, which may be administered as disclosed herein. Following administration of vaccine composition or one or more polynucleotides, the subject may be protected against CDI or rCDI.

A disclosed vaccine composition may be administered in a therapeutically effective amount. The terms “effective amount” or “therapeutically effective amount” refer to an amount of an antigen or vaccine that would induce an immune response in a subject receiving the antigen or vaccine which is adequate to prevent signs or symptoms of disease, including adverse health effects or complications thereof, caused by infection with a pathogen, such as a virus or a bacterium. Humoral immunity or cell mediated immunity or both humoral and cell mediated immunity may be induced. The immunogenic response of an animal to a vaccine may be evaluated, e.g., indirectly through measurement of antibody titers, lymphocyte proliferation assays, or directly through monitoring signs and symptoms after challenge with wild-type strain. The protective immunity conferred by a vaccine may be evaluated by measuring, e.g., reduction in clinical signs such as mortality, morbidity, temperature number, overall physical condition, and overall health and performance of the subject. The amount of a vaccine that is therapeutically effective may vary depending on the particular vaccine used, or the condition of the subject, and may be determined by a physician.

Methods of determining suitable dosage or dosage ranges for individual treatment are known to those in the art. For methods provided herein, a disclosed vaccine composition or polynucleotide encoding the composition or its components can be administered by any means that achieves the intended purpose or is deemed appropriate by those skilled in the art. In some embodiments, the vaccine composition is administered either as a single dose, at least two doses, or, when appropriate, as continuous administration using, for instance, a mini pump system. In some cases, the vaccine is provided as a liquid dosage form, or as a lyophilized dosage form that is, for example, reconstituted prior to administration.

The vaccine composition may be administered using any suitable route of administration, including, but are not limited to, direct injection. In certain embodiments, each dose is administered intramuscularly.

In another aspect, methods are provided for preventing or reducing attachment of C. difficile to an epithelial cell in a subject in need thereof, comprising delivering or administering the vaccine composition described herein to the subject. The terms “subject,” “patient,” and “individual,” may be used interchangeably in this disclosure and refer to all animals, including mammals, e.g., a human or non-human, who are susceptible to, prone to, or suffering from an indicated disease, disorder, or condition, and who may be treated with a pharmaceutical compositions or in accordance with the disclosed methods. In some embodiments, the subject is a mammal, e.g., a human or a non-human mammal. A subject “in need of” treatment according to the present disclosure may be “susceptible to,” suffering from,” or “suspected of suffering from” a disease, disorder, or condition (for example C. difficile infection) may have been positively diagnosed or otherwise presents with a sufficient number of risk factors or a sufficient number or combination of signs or symptoms a disease or disorder. Thus, subjects susceptible to, suffering from, and suspected of suffering from, a specific disease or disorder are not necessarily distinct groups.

In embodiments, a subject may be susceptible to a C. difficile infection. In embodiments, C. difficile may be present in the subject (or a subject is infected with a C. difficile). The C. difficile present in the subject may be in the form of vegetative cells, germinating cells, spore cells, or combinations thereof. Due to the strictly anaerobic nature of the vegetative form, spores are the main morphotype (or type or phenotype of the cell) in infection and transmission of the disease. Under suitable conditions, C. difficile spores will germinate and produce the pathogenic vegetative form. The subject may be administered the vaccine composition at a time before C. difficile is present in the subject, at a time after spore cells are present in the subject, and/or at a time after vegetative cells are present in the subject.

As used herein, the term “reducing” refers to a decrease of C. difficile attachment to an epithelial cell. The decrease can be in comparison to a suitable control. In some embodiments, a suitable control is a subject who has C. difficile present in their body, but the subject has not been administered the vaccine composition. The attachment of C. difficile to epithelial cells may be reduced by at least 5%, 10%, 15%, 20%, 25%, 30%, 35%, 40%, 45%, 50%, 55%, 60%, 65%, 70%, 75%, 80%, 85%, 90%, 95%, or 99%.

In some embodiments, the method further comprises targeting germinating C. difficile cells and/or C. difficile spore cells. In some embodiments, the method further comprises transitioning the spore C. difficile cells to germinating C. difficile cells. The spores can be transitioned to germinating cells by treatment with antibiotics. As the inventor describes in the Examples, antibiotic treatment can result in an overall decrease in gut microflora. The overall decrease in gut microflora can cause spore cells to transition to germinating cells due to reduced competition for nutrients. Therefore, in some embodiments, the method further comprises administering an antibiotic to the subject. The antibiotic may be administered after the vaccine composition is administered. In embodiments, the antibiotic is administered after C. difficile is present in the subject.

In some embodiments, the vaccine composition reduces attachment of C. difficile to a cholesterol-rich lipid raft(s) or other attachment point(s) in the membrane of the epithelial cell. As the inventor describes in the Examples, CDT and SLP facilitate the attachment of C. difficile to lipid raft(s) or other attachment point(s) in the membrane of epithelial cells. The CDT binary protein is responsible for colonization, invading host cells, disrupting their actin cytoskeleton, leading to cell leakage and death. Administering the vaccine composition to the subject builds immunity in the subject to CDTa, CDTb, HMW SLP, and/or LMW SLP. The subject's immune system can then prevent attachment of C. difficile to the cholesterol-rich lipid raft(s) or other attachment point(s) in the membrane of the epithelial cells.

In another aspect, disclosed herein are methods of preventing and treating diseases, disorders, or conditions caused by infection of C. difficile comprising delivering or administering the vaccine composition described herein to a subject in need thereof. As used in this disclosure, the terms “disease,” “disorder,” and “condition” are used interchangeably. In embodiments, the disease, disorder, or condition is selected from the group consisting of chronic diarrhea, high cholesterol, heart attack, stroke, irritable bowel syndrome, metabolic dysfunction-associated steatoic liver disease, metabolic dysfunction-associated steatohepatitis, and colorectal cancer. The inventor describes in the examples how each of these conditions is associated with C. difficile infection. In some embodiments, the subject is a human.

The vaccine composition may be delivered or administered to a subject before C. difficile infection, that is before C. difficile is present in the subject. The vaccine may be administered after C. difficile is present in the subject but before manifestation of a condition (for example, show symptoms or other evidence of symptoms) associated with C. difficile infection. The vaccine composition may be administered after manifestation of a condition caused by infection of C. difficile, such as chronic diarrhea. The vaccine composition may also be administered before recurrence of a condition caused by C. difficile infection, such as chronic diarrhea, high cholesterol, heart attack, stroke, irritable bowel syndrome, or liver disease.

The vaccine can be administered in early childhood with possible inoculations to older populations, including by potential booster(s). For example, if not previously vaccinated, a woman that is planning to have children may be vaccinated prior to pregnancy. The vaccine has precedent per other similar inactivated protein vaccines accepted globally, including the DPT vaccine (Diphtheria (Corynebacterium diphtheriae), Whooping Cough (Bordetella pertussis), and Tetanus (Clostridium tetani)).

In some embodiments, the vaccine is delivered or administered as a series of administrations to a subject, for example a human, for example at 2 months post-birth, 4 months post-birth, 6 months post-birth, 15 months post-birth, 16 months post-birth, 17 months post-birth, and 18 months post-birth, and any time 4 years through 6 years post-birth. A booster may be administered at 11 to 12 years post-birth. A booster may be administered every 10 years thereafter or post-birth.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

### Illustrative Embodiments

Embodiment 1. A vaccine composition comprising at least two polypeptides selected from the group consisting of a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP).

Embodiment 2. The vaccine composition of embodiment 1, wherein the at least two polypeptides are CDTb and HMW SLP.

Embodiment 3. The vaccine composition of embodiment 1 or 2, wherein the amino acid sequence of CDTb is selected from the group consisting of SEQ ID NOs: 10-19.

Embodiment 4. The vaccine composition of any one of the preceding embodiments, wherein the amino acid sequence of HMW SLP comprises SEQ ID NO: 26.

Embodiment 5. The vaccine composition of any one of the preceding embodiments, further comprising CDTa, wherein the amino acid sequence of CDTa is selected from the group consisting of SEQ ID NOs: 1-9.

Embodiment 6. The vaccine composition of any one of the preceding embodiments, further comprising LMW SLP, wherein the amino acid sequence of the LMW SLP comprises SEQ ID NOs: 23 or 24.

Embodiment 7. The vaccine composition of any one of the preceding embodiments, further comprising an adjuvant.

Embodiment 8. A fusion protein comprising the vaccine composition of any one of the preceding embodiments.

Embodiment 9. A pharmaceutical composition comprising (i) the vaccine composition of any one of embodiments 1-6 and (ii) a suitable pharmaceutical carrier.

Embodiment 10. A polynucleotide encoding the vaccine composition of any one of embodiments 1-6.

Embodiment 11. The polynucleotide of embodiment 10, wherein the polynucleotide is codon-optimized for expression of the vaccine composition.

Embodiment 12. An expression vector comprising a promoter operably linked to the polynucleotide of embodiment 10 or 11.

Embodiment 13. A virus comprising the polynucleotide of embodiment 10 or 11.

Embodiment 14. A modified cell comprising the vaccine composition of any one of embodiments 1-7, the pharmaceutical composition of embodiment 9, the polynucleotide of embodiment 10 or 11, the expression vector of embodiment 12, or the virus of embodiment 13.

Embodiment 15. A method of preparing the vaccine composition of any one of embodiments 1-7, comprising expressing a polynucleotide encoding the at least two polypeptides, or the polynucleotide of embodiment 10 or 11.

Embodiment 16. The method of embodiment 15, further comprising introducing the polynucleotide into a cell, optionally a mammalian cell.

Embodiment 17. The method of embodiment 15 or 16, further comprising purifying the at least two polypeptides.

Embodiment 18. The method of any one of embodiments 15-17, further comprising inactivating the at least two polypeptides.

Embodiment 19. The method of embodiment 18, wherein the at least two polypeptides are inactivated by formaldehyde or a solution comprising formaldehyde.

Embodiment 20. The method of any one of embodiments 15-19, wherein the at least two polypeptides are CDTb and HMW SLP.

Embodiment 21. The method of any one of embodiments 15-20, wherein the amino acid sequence of CDTb is selected from the group consisting of SEQ ID NOs: 10-19.

Embodiment 22. The method of any one of embodiments 15-21, wherein the amino acid sequence of HMW SLP comprises SEQ ID NO: 26.

Embodiment 23. The method of any one of embodiments 15-22, further comprising CDTa, wherein the amino acid sequence of CDTa is selected from the group consisting of SEQ ID NOs: 1-9.

Embodiment 24. The method of any one of embodiments 15-23, further comprising LMW SLP, wherein the amino acid sequence of the LMW SLP comprises SEQ ID NOs: 23 or 24.

Embodiment 25. A method of preventing or reducing attachment of Clostridioides difficile (C. difficile) to an epithelial cell in a subject in need thereof comprising administering the vaccine composition of any one of embodiments 1-7 to the subject, the pharmaceutical composition of embodiment 9, the polynucleotide of embodiment 10 or 11, the expression vector of embodiment 12, or the virus of embodiment 13.

Embodiment 26. The method of embodiment 25, further comprising targeting germinating C. difficile cells and/or spore C. difficile cells.

Embodiment 27. The method of embodiment 26, further comprising transitioning the spore C. difficile cells to germinating C. difficile cells.

Embodiment 28. The method of embodiment 25, wherein the vaccine composition reduces attachment of C. difficile to a cholesterol-rich lipid raft(s) or other attachment point(s) in the membrane of the epithelial cell.

Embodiment 29. The method of embodiment 25, wherein the vaccine composition is administered before C. difficile infection in the subject.

Embodiment 30. The method of embodiment 25, further comprising administering an antibiotic to the subject.

Embodiment 31. A method of preventing or treating a disease, disorder, or condition caused by infection of Clostridioides difficile comprising administering the vaccine composition of any one of embodiments 1-7, the pharmaceutical composition of embodiment 9, the polynucleotide of embodiment 10 or 11, the expression vector of embodiment 12, or the virus of embodiment 13.

Embodiment 32. The method of embodiment 31, wherein the disease, disorder, or condition is selected from the group consisting of chronic diarrhea, high cholesterol, heart attack, stroke, irritable bowel syndrome, metabolic dysfunction-associated steatoic liver disease, metabolic dysfunction-associated steatohepatitis, and colorectal cancer.

Embodiment 33. The method of embodiment 31, wherein the disease, disorder, or condition is chronic diarrhea.

Embodiment 34. The method of embodiment 31, wherein the subject is a human.

Embodiment 35. A method of vaccinating a subject, comprising administering to the subject the vaccine composition of any one of embodiments 1-7, the pharmaceutical composition of embodiment 9, the polynucleotide of embodiment 10 or 11, the expression vector of embodiment 12, or the virus of embodiment 13.

Embodiment 36. The vaccine composition of any one of embodiments 1-7, the pharmaceutical composition of embodiment 9, the polynucleotide of embodiment 10 or 11, the expression vector of embodiment 12, or the virus of embodiment 13 for use in preventing or treating a disease, disorder, or condition caused by infection of Clostridioides difficile, wherein the disease, disorder, or condition may be selected from the group consisting of chronic diarrhea, high cholesterol, heart attack, stroke, irritable bowel syndrome, metabolic dysfunction-associated steatoic liver disease, metabolic dysfunction-associated steatohepatitis, and colorectal cancer.

Embodiment 37. Use of the vaccine composition of any one of embodiments 1-7, the pharmaceutical composition of embodiment 9, the polynucleotide of embodiment 10 or 11, the expression vector of embodiment 12, or the virus of embodiment 13 in manufacturing a medicament for preventing or treating a disease, disorder, or condition caused by infection of Clostridioides difficile, wherein the disease, disorder, or condition may be selected from the group consisting of chronic diarrhea, high cholesterol, heart attack, stroke, irritable bowel syndrome, and colorectal cancer.

### Examples

Clostridioides difficile (C. difficile) is ubiquitous in the environment and on food. It is especially common as a spore forming bacteria which is resistant to high cooking temperatures. C. difficile lives primarily in the human colon section of the intestines, attached to the epithelial cell lipid raft(s) or other attachment point(s) in the membrane by both C. difficile transferase (CDT, or binary toxin CDT), namely the proteins CDTa (about 463 amino acids) and CDTb (about 876 amino acids), as well as surface layer proteins (SLPs). The surface layer proteins contain a conserved high molecular weight and a highly variable low molecular weight SLP. All of these proteins may cause diarrhea, but the two main pathogenic toxins of C. difficile, are the proteins, Toxin A (308 kDa) and Toxin B (270 kDa).

C. difficile attaches to host cells at the boundary of cholesterol-rich lipid raft(s) or other attachment point(s) in the membrane and the normal membrane. The greater the number of lipid raft(s) or other attachment point(s) in the membrane, with their high cholesterol content, the easier it is for C. difficile to attach. In host cell membranes, which are mainly composed of non-polar saturated fatty acid regions, the more polar regions of unsaturated and polyunsaturated fatty acids adjacent to cholesterol are the preferred sites for the attachment of C. difficile's polar peptides and proteins.

C. difficile infection (CDI) is a serious bacterial infection which causes inflammation of the colon, leading to symptoms like diarrhea, fever, abdominal pain, and in severe cases, life-threatening complications. C. difficile infections are one of the most common healthcare associated infections in the US and cause roughly 500K infections annually. The CDC estimates that 30K deaths are directly attributable to the disease and annual costs exceed $5 billion, including expenses related to hospitalization, treatment, and lost productivity.

Antibiotic use is the common treatment for C. difficile, but paradoxically can lead to an increase in CDI, by reducing overall gut microflora. This dysbiosis can lead dormant C. difficile populations that have colonized the colon (i.e. the spore form) to shift to vegetative and germinating C. difficile cells, which take advantage of the reduced competition for nutrients. When they germinate, the C. difficile cells produce Toxin A and B, as well as additional CDT and SLP proteins, which result in increased binding of more C. difficile cells to epithelial lipid raft(s) or other attachment point(s) in the membrane.

Two recent major Phase III clinical trials by pharmaceutical companies have been conducted against C. difficile Toxin A and Toxin B. Each clinical trial was conducted with vaccine candidates against these toxins, the two primary causes of C. difficile-caused chronic diarrhea. However, neither of these trials met their clinical endpoints, and both were abandoned. These clinical trials and vaccine candidates failed to treat the C. difficile colonization problem in the colon. Thus, after (1) antibiotic treatment destroyed most vegetative bacteria, (2) C. difficile spores remained to become vegetative cells again under ideal conditions, Toxin A and Toxin B eventually were produced again, and pathogenesis was restored. These vaccines did not stop re-infections because they did not get rid of the spores. Recent vaccine candidates of relevance as in Pfizer Patent Publication No. US20220160859A1 have targeted the Clostridioides difficile Toxins A and/or B only and are inactivated versions/variants of those toxins. Standard industry techniques were used to create and administer those vaccines during clinical trials. To the inventor's knowledge, there are no candidate vaccines for C. difficile meant to lower cholesterol, reducing the risk of heart attack and stroke.

Unlike the previous vaccine candidates, the vaccine provided in this disclosure targets both the CDT and SLP binding proteins that are integral to C. difficile binding to the colon epithelial cells, as well as C. difficile's colonization and spore formation in the colon. This new vaccine will target the root cause of CDI and rCDI, the primary cause of chronic diarrhea, as well as related diseases, disorders, or conditions such as high cholesterol, heart attack, stroke, irritable bowel syndrome, and colorectal cancer.

A vaccine will be developed that prevents and/or reduces attachment of C. difficile to host epithelial cells at the cholesterol and sphingolipid-rich microdomains of the host cell membrane, known as lipid raft(s) or other attachment point(s) in the membrane. lipid raft(s) and other attachment point(s) in the membrane are important for various cellular processes, including signal transduction and cell adhesion. C. difficile attaches to host cells at the boundary of these cholesterol-rich lipid raft(s) and other attachment point(s) in the membrane and the normal membrane. The vaccine targets two protein groups that are involved in attachment to the lipid raft(s) and other attachment point(s) in the membrane region. The first protein group is C. difficile transferase binding proteins (CDT), composed of inactivated CDTa and CDTb. These proteins produce a binary toxin specific to C. difficile that is known as the 3^{rd} toxin of the bacterium. CDTb is known to attach to lipid raft(s) or other attachment point(s) in the membrane, facilitating the entry of CDTa into the host cell. The CDT binary protein is responsible for colonization, invading host cells, disrupting their actin cytoskeleton, leading to cell leakage and death. The second protein group is surface layer proteins (SLPs), which are the most predominant proteins on the vegetative C. difficile surface. SLPs bind to the lipid raft(s) or other attachment point(s) in the membrane as well, play important roles in C. difficile colonization and induce inflammasome activation. C. difficile SLPs contain a conserved high molecular weight and a highly variable low molecular weight SLP. As the vaccine would greatly reduce the bacteria's attachment, this would also minimize the release of C. difficile's pathogenic Toxins A and B into host cells, reducing or preventing diseases or conditions caused by C. difficile, which are described below. This will lead to prevention and/or reduction in severity of C. difficile infection (CDI) and/or recurrent infection (rCDI).

This vaccine targets two protein groups: CDT, namely the CDTa and CDTb proteins of about 463 amino acids in length and about 876 amino acids in length respectively, which together produce a binary toxin (known as the 3^{rd} toxin of C. difficile), as well SLPs, a conserved high molecular weight and a highly variable low molecular weight SLP. The composition of the vaccine can contain inactivated versions of these proteins, created and purified using industry-standard techniques that are in common with similar vaccines and similar bacteria, such as that of the Tetanus (Clostridium tetani) vaccine, which is created from inactivated tetanus toxin (a protein of 150 kDa in size). The Tetanus vaccine is used worldwide, has been used for almost a century, and has a long-term safety and efficacy record.

The vaccine can be administered in early childhood with possible inoculations to older populations by potential booster(s). If not previously vaccinated, a woman that is planning to have children should be vaccinated prior to pregnancy. The vaccine has precedent per other similar inactivated protein vaccines accepted globally, including the DPT vaccine, for Diphtheria (Corynebacterium diphtheriae), Whooping Cough (Bordetella pertussis), and Tetanus (Clostridium tetani).

Rather than creating a vaccine pointed at reducing Toxin A and Toxin B, (the pathogenic toxins of C. difficile), this patent will reduce the population of C. difficile in the colon by targeting the proteins that bind C. difficile to lipid raft(s) or other attachment point(s) in the membrane, which leads to colonization. Previous “unsuccessful” vaccines were targeted at reducing Toxin A and Toxin B only. In those studies, when the population of C. difficile spores was large, spores remained after treatment, C. difficile continued to germinate and continued producing Toxin A and Toxin B. The vaccine described in this patent will reduce the population of C. difficile in the colon by targeting the CDT and SLP proteins. As C. difficile uses CDT and SLP proteins to obtain nutrients from the colon cells, these proteins are a more logical and effective vaccine target than vaccines targeted at Toxin A and Toxin B.

### Diseases Targeted by the Vaccine

### Chronic Diarrhea

The vaccine composition can greatly reduce the bacteria's attachment to and in a cell, which can also minimize the release of C. difficile's pathogenic Toxins A and B into host cells. These effects of the vaccine composition can lead to prevention and/or reduction in severity of C. difficile infection and/or recurrent infection (rCDI). CDI and rCDI are primary causes of chronic diarrhea, and chronic diarrhea is the most common disease associated with CDI and rCDI. CDI is a serious bacterial infection that causes inflammation of the colon, leading to symptoms like diarrhea, fever, abdominal pain, and in severe cases, life-threatening complications. C. difficile infections are one of the most common healthcare associated infections in the U.S. and cause roughly 500,000 infections annually. The CDC estimates that 30,000 deaths are directly attributable to the disease and annual costs exceed $5 billion, including expenses related to hospitalization, treatment, and lost productivity.

### Cholesterol and Heart Attack and Stroke

Heart attack and stroke (HAST) kills more U.S. citizens than any other disease. The case numbers of heart attacks and stroke exceed 1.6M annually, resulting in more than 500,000 deaths, at a cost exceeding $300B per year. This cost represents the largest portion of the $422.3B spent on cardiovascular diseases (Table 1; American Heart Association's 2024 Heart Disease and Stroke Statistics Update Fact Sheet).

TABLE 1
Heart Attack and Stroke Facts

| | | | Annual Associated |
|---|---|---|---|
| Diseases | Annual Cases | Annual Deaths | Costs |
| Heart attack | 805,000 | 375,000 | Estimated >$300B |
| Stroke | 795,000 | 163,000 | |

It is generally accepted that a significant cause of heart attack and stroke is elevated cholesterol levels, particularly low-density lipoprotein (LDL) cholesterol which can build up on the inner walls of one's arteries. This buildup, along with other substances like calcium and fat, forms a plaque called atherosclerosis. As the plaque accumulates, it narrows the arteries, can restrict blood flow and can also burst leading to a blood clot that can travel to arteries of the heart, leading to a heart attack, or can travel to the arteries in the brain, leading to a stroke. Per expert guidance, high levels of cholesterol are deemed to be above 200 mg/dl and above 130 mg/dl of LDL cholesterol. Optimal levels are below 150 mg/dl and below 100 mg/dl of LDL cholesterol. According to the CDC, more than 102M Americans in the US have high cholesterol.

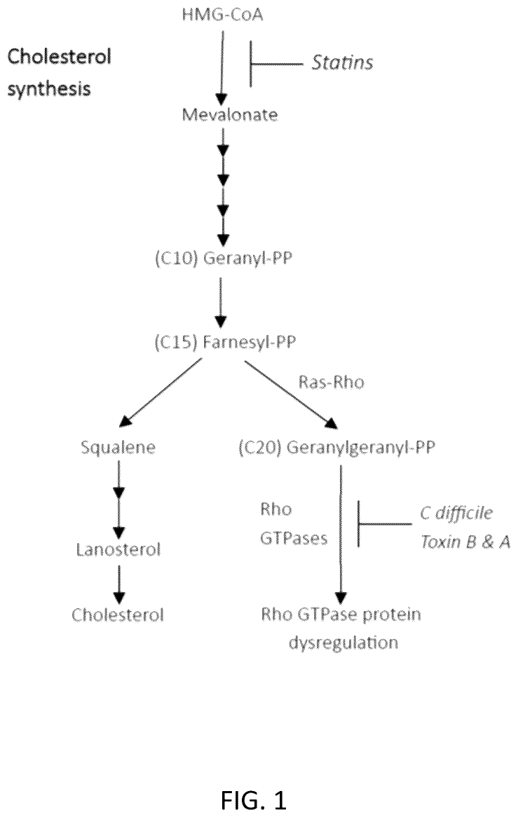

Cholesterol comes from the human body synthesizing cholesterol in the liver (see, FIG. 1) as well as from a diet containing animal meat. Having recognized that this metabolic pathway may lead to elevated cholesterol, pharmaceutical products (e.g., statins) were widely introduced in the 1980s to block this pathway and lower cholesterol. The annual cost of statin drugs in the United States to the healthcare system is around $24.5B [Lin, 2021], and as with most other pharmaceutical products, statins have negative side effects. As shown in FIG. 1, statins are cholesterol-lowering drugs that inhibit the mevalonate pathway by blocking the enzyme HMG-CoA reductase (HMG-CoA). Statin drugs can lower elevated LDL cholesterol by up to 40%±10%, but this is per using higher dosages of statins or more potent candidates with the most side effects. The average plasma LDL cholesterol lowering capability of these drugs is 20-30%. While greatly beneficial, the side-effects of statin's inhibition of the mevalonate pathway can lead to several negative downstream effects by blockage of the body's important Ras and Rho signaling pathways. Two of the most well-documented side effects of statins are muscle fatigue symptoms and new-onset type 2 diabetes mellitus [Ward, 2019].

The relationship between colonic bacteria and cholesterol has been demonstrated in several studies, including most recently by the lowering of elevated LDL cholesterol by 23%±5% during treatment with oral antibiotics [Jenkins, 2005]. When the antibiotics were no longer administered, cholesterol elevated above baseline levels, then returned to baseline. This data strongly suggests the involvement of a spore-forming bacteria in the antibiotic-associated cholesterol shifts, and a bacterium that has impacts within the cholesterol metabolic pathway (FIG. 1). A bacterium that fits this model is C. difficile. When expressed and secreted within the colon during germination, C. difficile produces Toxins A and B (308 kDa and 270 kDa respectively), which bind host cell receptors, are endocytosed by host cells and dysregulate Rho-family GTPases via glucosylation. Dysregulation of the Ras-Rho metabolic pathway shifts the farnesyl pyrophosphate (farnesyl-PP) pathway towards cholesterol production, resulting in elevated cholesterol when Toxins A and B are present. Given that C. difficile vegetative cells and Toxins A and B production are mostly eliminated during antibiotic administration, this would result in a normal functioning Ras-Rho pathway, and a lower cholesterol level. However, although antibiotics provide good research information, they are not a logical means to control cholesterol, reduce heart attack and stroke risk.

Other than antibiotics, a useful way to dramatically reduce Toxins A and B is to reduce their production by reducing C. difficile colonization in the colon. Colonization and subsequent germination can be reduced by preventing the proteins CDT and SLP from binding to the epithelial lipid raft(s) or other attachment point(s) in the membrane via a vaccine that disrupts this binding. By disrupting this binding, C. difficile Toxins A and B can be reduced significantly, which would also reduce elevated LDL-cholesterol (without the side effects and high cost of statins). Subsequently, this would reduce the 500,000 annual heart attack and stroke deaths. It would also have the beneficial side effect of reducing the 30,000 annual deaths from the leading source of chronic diarrhea, C. difficile.

This vaccine has a competitive advantage over statins in that statins are expensive insufficient numbers of people take them, and statins have many side effects. This vaccine invention will be inexpensive, should have few negative side effects reduces/prevents colonization of the colon by C diff, reduces/prevents high LDL cholesterol and reduces/prevents the risk of heart attack and stroke.

### Irritable Bowel Syndrome

The Cleveland Clinic and American College of Gastroenterology (ACG) estimates that about 10-15% of the US population has irritable bowel syndrome (IBS). Some reports indicate that the total annual cost of IBS-related healthcare expenses and lost productivity in the United States exceeded $20 billion. There are a host of treatments for IBS aimed at reducing symptoms. Options include fiber supplements and laxatives for constipation (IBS-C), antidiarrheals and certain antibiotics (like rifaximin) for diarrhea (IBS-D), and antispasmodics for pain. Low-dose antidepressants can also help manage pain and gut function across IBS subtypes.

The relationship between C. difficile and post-infection IBS has been demonstrated in several studies. C. difficile produces potent toxins, primarily Toxin A and Toxin B, and sometimes a third toxin called C. diff transferase (CDT). These toxins directly damage the cells lining the colon (colonic epithelial cells). They disrupt the tight junctions between these cells, leading to increased intestinal permeability (often referred to as “leaky gut”). This damage causes inflammation and can lead to cell death. The damage caused by C. difficile toxins triggers a significant inflammatory response in the gut. The toxins stimulate the affected colonic epithelial cells to release pro-inflammatory cytokines and chemokines, which recruit immune cells, particularly neutrophils, to the site of infection. This acute inflammation is a hallmark of colitis. Persistent low grade inflammation, even after the initial C. difficile infection clears can cause irritable bowel syndrome (IBS) in about 25% of patients (post-IBS). C. difficile infection itself often occurs after antibiotic use, which profoundly disrupts the natural balance of the gut microbiota (dysbiosis). The toxins further contribute to this imbalance by creating an environment conducive to C. difficile overgrowth and potentially altering the composition and function of the remaining gut microbes. This sustained dysbiosis can contribute to ongoing gastrointestinal symptoms characteristic of IBS. There's growing evidence that C. difficile toxins and the resulting inflammation can affect the enteric nervous system, often called the “second brain” of the gut. Damage to enteroglial cells (support cells in the enteric nervous system) initiated by toxins is a proposed mechanism for the long-term gut dysfunction seen in PI-IBS. This can lead to altered gut motility (how food moves through the digestive tract) and visceral hypersensitivity (increased pain perception from the gut), both key features of IBS. Inflammation can also lead to the activation and degranulation of mast cells in the gut. Mast cells release various mediators that can contribute to visceral hypersensitivity and altered gut motility, further contributing to IBS symptoms. In essence, C. difficile toxins initiate a severe disruption of the gut's normal functioning through direct cellular damage, inflammation, and alterations to the gut microbiome and nervous system. While the acute infection resolves for many, these initial insults can trigger lasting changes that manifest as the chronic symptoms of IBS for about 25% of patients [Madan, 2012; Wadhwa, 2016; Taghaddos, 2024].

While antibiotics are a useful way to control vegetative C. difficile, they are not a long-term solution because they kill off all the beneficial bacteria, the C. difficile spores remain and then this leads to C. difficile further colonizing the gut. Other than antibiotics, a useful way to dramatically reduce Toxins A and B is to reduce their production by reducing C. difficile colonization in the colon. Colonization and subsequent germination of C. difficile spores can be reduced by preventing the proteins CDT and SLP from binding to the epithelial lipid raft(s) or other attachment point(s) in the membrane via a vaccine that disrupts this binding.

By disrupting this binding, C. difficile Toxin A and Toxin B can be reduced significantly, which would also reduce the 10-15% incidence of IBS in the US. It would also have the beneficial side effect of reducing the 30,000 annual deaths from the leading source of chronic diarrhea, C. difficile. This vaccine has a competitive advantage over current IBS treatments in that treatments can be expensive and must be taken daily. This vaccine invention will be inexpensive, should have few negative side effects, reduces/prevents colonization of the colon by C. difficile, and reduces/prevents IBS.

### Metabolic Dysfunction-Associated Steatoic Liver Disease and Metabolic Dysfunction-Associated Steatohepatitis

According to recent estimates, the prevalence of metabolic dysfunction-associated steatotic liver disease (MASLD) in the US is between 25-38% of adults and its prevalence has more than doubled over the past two decades. Metabolic dysfunction-associated steatohepatitis (MASH), a more severe form of MASLD, is estimated to affect about 5% of U.S. adults. MASH is becoming a leading cause of liver transplants. The total annual economic costs of MASLD and MASH due to healthcare expenses and lost productivity in the U.S. is well over $100 billion.

The current treatments for MASLD and its more severe form, MASH, focus on addressing the underlying metabolic issues and preventing disease progression. The cornerstone of treatment for both conditions is lifestyle modification. Although pharmacological treatments are emerging, they are expensive and require ongoing treatment.

The relationship between C. difficile, MASLD and MASH has been demonstrated in several studies. Toxin A and Toxin B, and other bacterial byproducts from C. difficile in the gut can directly contribute to liver damage via the “gut-liver axis”. This can lead to altered liver lipid metabolism, oxidative stress, and a buildup of metabolites indicative of liver damage. These metabolic changes are the primary drivers of steatosis and inflammation, which are the hallmarks of MASLD and the more progressive state of this disease, MASH.

C. difficile infection triggers significant metabolic and genetic changes in the liver, leading to a pro-inflammatory and pro-steatotic state that mirrors the characteristics of MASLD and MASH. Through a combination of liver metabolomics, transcriptomics, and gut metagenome analysis, it has been determined that C. difficile infections disrupt crucial metabolic pathways in the liver, including those related to lipid synthesis, breakdown, and signaling. In addition, there is an upregulation of genes and metabolites linked to lipid accumulation and inflammation, and a simultaneous decrease in antioxidant defense mechanisms, evidenced by a depletion of glutathione. These changes have been attributed to the infection itself, rather than any antibiotic treatments preceding it.

The “gut-liver” axis is central to this process. C. difficile-induced gut dysbiosis and a “leaky gut” allow Toxin A, Toxin B, and bacterial byproducts to reach the liver, causing inflammation and oxidative stress. It is proposed that a single acute episode of C. difficile infection is a risk factor for MASLD and MASH, by creating a foundation for metabolic dysfunction. It is also apparent that this is a “bidirectional vicious cycle,” between C. difficile infection and liver disease, where pre-existing liver disease increases the risk of C. difficile infection, and the infection, in turn, accelerates the progression of the liver disease. In summary, for those individuals without any liver disease, the presence of C. difficile infection can lead to MASLD. For those individuals who already have MASLD, the presence of C. difficile can lead to disease progression of MASLD to MASH [Lee, 2020; Yousefi, 2025].

While antibiotics are a useful way to control vegetative C. difficile, they are not a long-term solution because they kill off many of the beneficial bacteria, the C. difficile spores remain and then this leads to C. difficile further colonizing the gut. Other than antibiotics, a useful way to dramatically reduce Toxins A and B is to reduce their production by reducing C. difficile colonization in the colon. Colonization and subsequent germination of C. difficile spores can be reduced by preventing the proteins CDT and SLP from binding to the epithelial lipid raft(s) or other attachment point(s) in the membrane via a vaccine that disrupts this binding.

By disrupting this binding, C. difficile Toxin A and Toxin B can be reduced significantly, which would also reduce the epidemic levels of MASLD and MASH in the U.S. It would also have the beneficial side effect of reducing the 30,000 annual deaths from the leading source of chronic diarrhea, C. difficile. This vaccine has a competitive advantage over current MASLD and MASH treatments in that treatments can be expensive and have to be taken on a routine basis. This vaccine invention will be inexpensive, should have few negative side effects, reduces/prevents colonization of the colon by C. difficile, and reduces/prevents MASLD and MASH.

### Colorectal Cancer

According to recent estimates, the prevalence of new colorectal cancer cases in the US is about 150,000 adults annually, and about 50,000 adults die from the disease annually. The associated direct and indirect costs are estimated at greater than $24 billion annually. Colorectal cancer treatment is a multi-faceted approach determined by the cancer's stage and location. Surgery is the most common method, often used to remove the tumor and surrounding tissue. Chemotherapy uses drugs to kill cancer cells and can be used before or after surgery, or to manage advanced disease. Targeted therapy and immunotherapy are newer treatments that specifically attack cancer cells or boost the body's immune response, offering hope for patients with specific tumor characteristics.

Recent studies have shown that human colorectal cancer-derived toxigenic C. difficile strains promote colorectal cancer tumors in mice. When C. difficile was removed from a tumorigenic bacterial mixture, the mice did not develop tumors. Conversely, adding these toxigenic C. difficile strains to a nontumorigenic slurry from a patient was enough to induce tumor formation. The bacterium induces Wnt signaling in colonic progenitor cells, a hallmark of colorectal cancer. It also leads to the production of reactive oxygen species, which can damage DNA and contribute to cancer. Furthermore, the presence of toxigenic C. difficile was associated with a pro-tumorigenic immune response in the gut, marked by the infiltration of myeloid cells and the production of IL17 from various immune cells. These immune and cellular changes appear to be directly driven by Toxin B, as mutant strains of C. difficile lacking Toxin B were unable to induce these effects or cause significant tumor formation. Thus, persistent exposure to the Toxin B produced by C. difficile is critical for promoting tumorigenesis. Long-term colonization with toxin-producing C. difficile is a risk factor for colorectal cancer in humans [Drewes, 2022; Anderson, 2023].

While antibiotics are a useful way to control vegetative C. difficile, they are not a long-term solution because they kill off many of the beneficial bacteria, the C. difficile spores remain and then this leads to C. difficile further colonizing the gut. Other than antibiotics, a useful way to dramatically reduce Toxins A and B is to reduce their production by reducing C. difficile colonization in the colon. Colonization and subsequent germination of C. difficile spores can be reduced by preventing the proteins CDT and SLP from binding to the epithelial lipid raft(s) or other attachment point(s) in the membrane via a vaccine that disrupts this binding.

By disrupting this binding, C. difficile Toxin A and Toxin B can be reduced significantly, which would also reduce colorectal cancer in the US. It would also have the beneficial side effect of reducing the 30,000 annual deaths from the leading source of chronic diarrhea, C. difficile. This vaccine has a competitive advantage over current colorectal cancer treatments in that this vaccine invention will be inexpensive, should have few negative side effects, reduces/prevents colonization of the colon by C. difficile, and reduces/prevents colorectal cancer.

### REFERENCES

1. American Heart Association [AHA] 2024 Heart Disease and Stroke Statistics Update Fact Sheet—www.heart.org
2. CDC Heart Disease Facts—www.cdc.gov
3. NFID, C diff: An Urgent Public Health Threat—www.nfid.org
4. Papatheodorou P, et. al. (2013). Clostridium difficile Binary Toxin CDT Induces Clustering of the Lipolysis-Stimulated Lipoprotein Receptor into Lipid Rafts. MBio. 4(3): 1-13. doi:10.1128/mBio.00244-13
5. Yu C, et. al. (2020). Membrane Cholesterol Is Crucial for Clostridium difficile Surface Layer Protein Binding and Triggering Inflammasome Activation. Frontiers in Immunol. 11: 1-9. doi:10.3389/fimmu.2020.01
6. Mori N, Takahashi T, (2018). Characteristics and Immunological Roles of Surface Layer Proteins in Clostridium difficile. Ann. Lab. Med. 38: 189-195. doi.org/10.3343/alm.2018.38.3.189
7. Kordus S, Thomas A, Lacy D (2022). Clostridioides difficile toxins: mechanisms of action and antitoxin therapeutics. Nat Rev Microbiol. 20(5): 285-298. doi:10.1038/s41579-021-00660-2
8. Lin S-Y, et. al. (2021). Trends in Use and Expenditures for Brand-name Statins After Introduction of Generic Statins in the US, 2002-2018. JAMA Network Open. 4(11):e2135371. doi:10.1001/jamanetworkopen.2021.35371
9. Ward N, Watts G, Eckel R (2019). Statin Toxicity Mechanistic Insights and Clinical Implications. Circulation Research. 124 (328-350). doi:10.1161/CIRCRESAHA.118.312782
10. Jenkins D, et. al. (2005). Effects of antibiotics as cholesterol-lowering agents. Metabolism: Clinical and Experimental. 54 (103-112). doi.org/10.1016/j.metabol.2004.07.019
11. Madan, R. et. al. (2012). Immune responses to Clostridium difficile infection. Trends Mol Med. 2012 Oct. 16; 18(11):658-666. doi.org/10.1016/j.molmed.2012.09.005
12. Wadhwa, A., et al. (2016). High risk of post-infectious irritable bowel syndrome in patients with Clostridium difficile infection. Aliment Pharmacol Ther. 2016 September; 44(6): 576-582. doi.org/10.1111/apt.13737
13. Taghaddos, D., et al. (2024). Post-infectious IBS following Clostridioides difficile infection; role of microbiota and implications for treatment. Dig. and Liver Dis. 56(11): 1805-1809. doi.org/10.1016/j.dld.2024.03.008
14. Lee, S., et al. (2020). Intestinal Clostridioides difficile Can Cause Liver Injury through the Occurrence of Inflammation and Damage to Hepatocytes. Biomed Research Intl. Volume 2020, Article ID 7929610: 1-10.
15. Yousefi, F., et. al. (2025). Clostridioides difficile infection induces a pro-inflammatory and pro-steatotic metabolic state in liver. NPJ Gut and Liver. 2, 5: 1-20.
16. Drewes, J., et. al. (2022). Human Colon Cancer-Derived Clostridioides difficile Strains Drive Colonic Tumorigenesis in Mice. Cancer Discov 2022; 12:1873-85. doi.org/10.1158/2159-8290.CD-21-1273
17. Anderson, S. & Sears, C. (2023). The Role ofthe Gut Microbiome in Cancer. A Review, With Special Focus on Colorectal Neoplasia and Clostridioides difficile. Clinical Infectious Diseases 2023; 77(S6):S471-8. doi.org/10.1093/Cid/ciad640
18. Maari, A. (2024). A systematic review of the economic burden of colorectal cancer. Health Sci. Rep. 2024; 7:e70002. doi.org/10.1002/hsr2.70002

Sequences

| SEQ ID NO: | Accession No. | Strain | Protein |
|---|---|---|---|
| 1 | AAF81760 | CCUG20309 | CDTa |
| 2 | ABS57476 | CD196 | CDTa |
| 3 | AEC11575.1 | AM478 | CDTa |
| 4 | AEC11569.1 | CD98 | CDTa |
| 5 | AEC11560.1 | AE16 | CDTa |
| 6 | AAB67304.2 | CD196 | CDTa |
| 7 | AEC11578.1 | A765 | CDTa |
| 8 | Q9KH42 | CCUG20309 | CDTa |
| 9 | Consensus Sequence | N/A | CDTa |
| 10 | AAF81761 | CCUG20309 | CDTb |
| 11 | ABS57477 | CD196 | CDTb |
| 12 | AEC11570.1 | CD98 | CDTb |
| 13 | AEC11585.1 | K744 | CDTb |
| 14 | AEC11576.1 | AM478 | CDTb |
| 15 | AEC11558.1 | AD667 | CDTb |
| 16 | AEC11564.1 | C421 | CDTb |
| 17 | AEC11579.1 | A765 | CDTb |
| 18 | AAB67305.1 | CD196 | CDTb |
| 19 | Consensus Sequence | N/A | CDTb |
| 20 | AJP12540.1 | 630 | SlpA |
| 21 | CAC35721.1 | R8366 | SlpA |
| 22 | CAC35720.1 | R7404 | SlpA |
| 23 | Consensus Sequence | N/A | LMW SLP (with |
| | | | linker) |
| 24 | Consensus Sequence | N/A | LMW SLP |
| | | | (without linker) |
| 25 | Consensus Sequence | N/A | Linker |
| 26 | Consensus Sequence | N/A | HMW SLP |

What is claimed:

1. A vaccine composition comprising at least two polypeptides selected from the group consisting of a Clostridioides difficile binary toxin B (CDTb), a Clostridioides difficile binary toxin A (CDTa), a High Molecular Weight portion of Clostridioides difficile Surface Layer Protein A (HMW SLP), and a Low Molecular Weight portion of Clostridioides difficile Surface Layer Protein A (LMW SLP).

2. The vaccine composition of claim 1, wherein the at least two polypeptides are CDTb and HMW SLP.

3. The vaccine composition of claim 2, wherein the amino acid sequence of CDTb is selected from the group consisting of SEQ ID NOs: 10-19.

4. The vaccine composition of claim 2, wherein the amino acid sequence of HMW SLP comprises SEQ ID NO: 26.

5. The vaccine composition of claim 2, further comprising CDTa, wherein the amino acid sequence of CDTa is selected from the group consisting of SEQ ID NOs: 1-9.

6. The vaccine composition of claim 2, further comprising LMW SLP, wherein the amino acid sequence of the LMW SLP comprises SEQ ID NOs: 23 or 24.

7. A pharmaceutical composition comprising (i) the vaccine composition of claim 1 and (ii) a suitable pharmaceutical carrier.

8. A polynucleotide encoding the vaccine composition of claim 1, wherein the polynucleotide is operably linked to a promoter.

9. The polynucleotide of claim 8, wherein the polynucleotide is codon-optimized for expression of the vaccine composition.

10. A modified cell comprising the vaccine composition of claim 1.

11. A method of preparing the vaccine composition of claim 1, comprising expressing a polynucleotide encoding the at least two polypeptides.

12. The method of claim 11, further comprising introducing the polynucleotide into a cell.

13. The method of claim 11, wherein the at least two polypeptides are CDTb and HMW SLP.

14. The method of claim 13, wherein the amino acid sequence of CDTb is selected from the group consisting of SEQ ID NOs: 10-19.

15. The method of claim 13, wherein the amino acid sequence of HMW SLP comprises SEQ ID NO: 26.

16. The method of claim 13, further comprising CDTa, wherein the amino acid sequence of CDTa is selected from the group consisting of SEQ ID NOs: 1-9.

17. The method of claim 13, further comprising LMW SLP, wherein the amino acid sequence of the LMW SLP comprises SEQ ID NOs: 23 or 24.

18. A method of preventing or reducing attachment of Clostridioides difficile (C. difficile) to an epithelial cell in a subject in need thereof comprising administering the vaccine composition of claim 1 to the subject.

19. The method of claim 18, further comprising targeting germinating C. difficile cells and/or spore C. difficile cells.

20. The method of claim 19, further comprising transitioning the spore C. difficile cells to germinating C. difficile cells.

21. The method of claim 19, wherein the vaccine composition reduces attachment of C. difficile to a cholesterol-rich lipid raft or other attachment point in the membrane of the epithelial cell.

22. A method of preventing or treating a disease, disorder, or condition caused by infection of Clostridioides difficile comprising administering the vaccine composition of claim 1.

23. The method of claim 22, wherein the disease, disorder, or condition is selected from the group consisting of chronic diarrhea, high cholesterol, heart attack, stroke, irritable bowel syndrome, metabolic dysfunction-associated steatoic liver disease, metabolic dysfunction-associated steatohepatitis, and colorectal cancer.

24. A method of vaccinating a subject, comprising administering to the subject the vaccine composition of claim 1.
